(19) United States
(12) Patent Application Publication
(10) Pub. No.: US 2006/0115901 A1
(43) Pub. Date: Jun. 1, 2006

(54) Method and media for single cell serum-free culture of CHO cells

(76) Inventors: Bahram Valamehr, Granada Hills, CA (US); Thomas Seewoester, Simi Valley, CA (US)

Correspondence Address: AMGEN INC.;LAW DEPARTMENT, 1201 AMGEN COURT WEST, SEATTLE, WA, 98119, US

(21) Appl. No.: 11/256,298

(22) Filed: Oct. 21, 2005

Related U.S. Application Data
(60) Provisional application No. 60/621,245, filed on Oct. 22, 2004.

(51) Int. Cl. C12N 5/00; C12N 5/02

(52) U.S. Cl. 435/404; 435/373

(57) ABSTRACT

The present invention relates to methods and media for single cell serum free culture of CHO cells. Generally, the invention relates to a method of culturing CHO cells at a cell density of less than 100 cells/ml in a serum-free cell culture medium. The medium is sufficient to support the growth of a single CHO cell and comprises a basal medium sufficient to support the growth of CHO cells and a supplemental medium, wherein the combined basal medium and supplemental medium comprise an antioxidant, L-glutamine, iron, ethanolamine, and albumin; and insulin, wherein the insulin may be present in either the basal medium or the supplemental medium, or both. Optionally, the medium may contain EGF or IGF.

### CROSS REFERENCE TO RELATED APPLICATION

This application claims the benefit of the filing date of U.S. Provisional Patent Application Serial No. 60/621,245, filed Oct. 22, 2004, the disclosure of which is incorporated, in its entirety, by this reference.

### FIELD OF INVENTION

The present invention relates to methods and cell culture media for serum-free single cell culture of Chinese hamster ovary (CHO) cells.

### BACKGROUND OF INVENTION

CHO cell lines are widely used for the expression of recombinant proteins, including recombinant antibodies. The development of a CHO cell line for production of a recombinant protein first requires that the selected cell population be homogeneous. This is achieved by dilution of an original cell population down to a single cell and then growing this single cell back to a larger cell population. The new cell population derived from the single cell is considered a clonal cell population or cell line and the process is termed “cell cloning”. The cell cloning process requires a special environment of correctly balanced nutrients specific for the extremely low cell density conditions. To create such an environment Fetal Bovine Serum (FBS) for Fetal Calf Serum (FCS), an animal derived raw material, is typically added to the cell culture medium to introduce the necessary nutrients and ensure a successful execution of the single cell cloning step. Serum, the fluid portion of blood which remains after removal of the blood corpuscles and fibrin, contains numerous biological components, including protein growth factors and nutrients, believed to be necessary for the growth and survival of cells. Engineered CHO cells (those in which a CHO cell line is transfected with a product gene and a selectable marker gene) are routinely grown in culture media containing serum. (References: J. Mol. App. Gen. 1981, 1, 165-175; Mol. & Cell Biol. 5, 1750-1759, 1985; PNAS 80 pp 4654-4658; Molecular and Cellular Biology 4, 166-172, 1984).

The use of serum in culture media, however, has significant disadvantages. Not only is serum prohibitively expensive, but serum is inherently uncharacterized, containing a large number of unknown and unquantified ingredients, the quality and quantity of which may be highly variable among different manufacturers and among different lots from a single manufacturer.

Consequently, much effort has been devoted to developing a serum-free culture medium that will support growth of CHO cells. Despite the desirability of eliminating serum from culture media, there exists significant impediments to the use of serum-free media for cell cloning. Past efforts to culture CHO cells in low density or single cell serum-free conditions have been largely unsuccessful. Use of serum-free medium results in very low initial cell cloning efficiency, and also typically fails to establish a stable clone in subsequent cell passages. It is widely believed that the removal of serum may remove important components that provide cell protection and detoxifying activity necessary for growth and expression of product. Serum is therefore widely considered to be an essential component of CHO cell media used at very low density cell conditions. Due to the inherently complex and uncharacterized nature of serum, however, the specific components of serum that are essential for low density culture of mammalian cells are unknown.

It is an object of the present invention to provide a culture medium for serum-free cell cloning that is capable of sustaining growth of CHO cells at very low cell density conditions, and which is equivalent or superior in cell cloning efficiency to media that includes serum.

### SUMMARY OF INVENTION

The present invention is directed to a novel biochemically defined serum-free CHO cell culture medium capable of supporting the growth of a single CHO cell.

In one aspect, the present invention is directed to a method of culturing a population of CHO cells at a cell density of less than about 100 cells/ml in a serum-free cell culture medium, comprising the steps of culturing a population of CHO cells at a cell density greater than about 10⁴ cells/ml in a basal medium sufficient to support the serum-free growth of CHO cells; reducing the CHO cell density to less than about 100 cells/ml; and adding to the basal medium a supplemental medium, wherein the combined basal medium and supplemental medium include an antioxidant, L-glutamine, iron, ethanolamine, recombinant albumin, and recombinant insulin, in an amount sufficient to support the growth of a single CHO cell. Optionally, the medium may contain one or more growth factors selected from the group consisting of recombinant epidermal growth factor and recombinant insulin like growth factor.

In another aspect, the present invention is directed to a method of culturing a single CHO cell in a serum-free cell culture medium, comprising the steps of culturing a population of CHO cells at a cell density greater than about 10⁴ cells/ml in a basal medium sufficient to support the serum-free growth of CHO cells; isolating a single CHO cell; and adding to the basal medium a supplemental medium, wherein the combined basal medium and supplemental medium include an antioxidant, L-glutamine, iron, ethanolamine, recombinant albumin, and recombinant insulin, in an amount sufficient to support the growth of a single CHO cell. Optionally, the medium may contain one or more growth factors selected from the group consisting of recombinant epidermal growth factor and recombinant insulin like growth factor.

In another aspect, the present invention is directed to a serum-free CHO cell culture medium comprising a basal medium sufficient to support the serum-free growth of CHO cells and a supplemental medium, wherein the combined basal medium and supplemental medium include an antioxidant, L-glutamine, iron, ethanolamine, recombinant albumin, and recombinant insulin, in an amount sufficient to support the growth of a single CHO cell.

A serum-free CHO cell culture medium comprising a basal medium sufficient to support the serum-free growth of CHO cells and a supplemental medium, wherein the combined basal medium and supplemental medium include an antioxidant, L-glutamine, iron,

ethanolamine, recombinant albumin, and recombinant insulin, and one or more growth factors selected from the group consisting of recombinant epidermal growth factor and recombinant insulin like growth factor, in an amount sufficient to support the growth of a single CHO cell.

In particular embodiments of the present invention, the basal medium has substantially the same ingredients as a basal medium selected from the group consisting of VM-Soy and 50/50, the antioxidant is present in a concentration of from about 0.01 mM to about 10 mM, the L-glutamine is present in a concentration of from about 2 mM to about 6 mM, the iron is present in a concentration of from about 10 mg/L to about 400 mg/L, the ethanolamine is present in a concentration of from about 1 mg/L to about 100 mg/L, the recombinant albumin is present in a concentration of from about 1 g/L to about 5 g/L, the recombinant insulin is present in a concentration of from about 10 g/L to about 20 g/L, the recombinant EGF is present in a concentration of from about 0.01 μg/L to about 100 μg/L, the recombinant IGF is present in a concentration of from about 1 μg/L to about 1500 μg/L.

In a more particular embodiment of the present invention, the antioxidant is present in a concentration substantially equivalent to a 1× solution of Sigma Cat. No. A-1345, the L-glutamine is present in a concentration of about 2 mM, the iron is present in concentration of about 200 mg/L, the ethanolamine is present in a concentration of about 30 mg/L, the recombinant albumin is present in a concentration of about 2 g/L, and the recombinant insulin is present in a concentration of about 15 mg/L, the recombinant EGF is present in a concentration of about 10 μg/L, and the recombinant IGF is present in a concentration of about 100 μg/L.

In yet another aspect, the present invention is directed to a CHO cell in a culture media as described above.

### BRIEF DESCRIPTION OF THE FIGURES

FIG. 1 shows the performance of various cell culture media on the growth of two CHO host cell lines, CS9 and AM-1/D, derived from DUXB11 cell line by adaptation to serum-free conditions and cloned. Performance is measured in terms of plating efficiency, which is the ratio of the number of wells in a 96-well plate that successfully produced a viable colony to that of the total number of wells seeded at one cell per well. As seen in FIG. 1, the addition of either formulation 1.0 or formulation 1.1 to the cloning media supports the growth of the single cell equally in comparison to that of the cloning media containing serum. Moreover, with no additions to the 50/50 cloning media there is no growth or survival detected. Thus, the data shows that single cell growth requires either the addition of serum or one of the formulations 1.0 or 1.1 to survive and proliferate.

FIG. 2 shows the effects of the addition and/or deletion of various key components on the growth and survival of the single cell colonies. As shown in FIG. 2, efficiency of cell growth is dramatically reduced by the addition of certain components, and is optimum using formulation 1.0 or 1.1 alone.

### DETAILED DESCRIPTION OF THE INVENTION

While the terminology used in this application is standard within the art, definitions of certain terms are provided herein to assure clarity and definiteness to the meaning of the claims.

Units, prefixes, and symbols may be denoted in their SI accepted form. Numeric ranges recited herein are inclusive of the numbers defining the range and include and are supportive of each integer within the defined range. Unless otherwise noted, the terms “a” or “an” are to be construed as meaning “at least one of.” The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present invention provides a biochemically defined serum-free cell culture medium and supplement for the culture and cloning of Chinese Hamster Ovary (CHO) cells. This formulation is unique in its ability to support the expansion of CHO cells at very low cell density conditions (e.g., less than 100 cells/ml). In particular, this formulation is unique in that it is capable of supporting the growth and expansion of a single CHO cell clone. The cell culture methods and media of the present invention are useful for and applicable to the culture of genetically engineered CHO cell lines used as hosts for the expression of recombinant proteins. The cell culture methods and media of the present invention are particularly useful for and applicable to the isolation and culture genetically engineered CHO cell clones, which are used as a basis for a CHO cell line that is then used as a host for the expression of clinical grade recombinant proteins.

Generally, the present invention is a method that utilizes a serum-free cell culture media for use in culturing CHO cells at very low cell density conditions. The present invention is also directed to a serum-free cell culture media for use in culturing CHO cells at very low cell density conditions. The present invention is also directed to CHO cells in a serum-free cell culture media, as described.

In one aspect, the present invention is directed to a method of culturing a population of CHO cells at a cell density of less than about 100 cells/ml in a serum-free cell culture medium, comprising the steps of culturing a population of CHO cells at a cell density greater than about 10⁴ cells/ml in a basal medium sufficient to support the serum-free growth of CHO cells; reducing the CHO cell density to less than about 100 cells/ml; and adding to the basal medium a supplemental medium, wherein the combined basal medium and supplemental medium include an antioxidant, L-glutamine, iron, ethanolamine, recombinant albumin, and recombinant insulin, in an amount sufficient to support the growth of a single CHO cell. Optionally, the medium may contain one or more growth factors selected from the group consisting of recombinant epidermal growth factor and recombinant insulin like growth factor. The ingredients of the basal medium and supplemental medium are present in the medium in concentrations and amounts that are physiologically acceptable for the particular density of the cells.

In another aspect, the present invention is directed to a method of culturing a single CHO cell in a serum-free cell culture medium, comprising the steps of culturing a population of CHO cells at a cell density greater than about 10⁴ cells/ml in a basal medium sufficient to support the serum-free growth of CHO cells; isolating a single CHO cell; and adding to the basal medium a supplemental medium, wherein the combined basal medium and supplemental medium include an antioxidant, L-glutamine, iron, ethanolamine, recombinant albumin, and recombinant insulin, in an amount sufficient to support the growth of a single CHO cell. Optionally, the medium may contain one or more growth factors selected from the group consisting of recombinant epidermal growth factor and recombinant insulin like growth factor. The ingredients of the basal medium and supplemental medium are present in the medium in concentrations and amounts that are physiologically acceptable for the particular density of the cells.

In another aspect, the present invention is directed to a serum-free CHO cell culture medium comprising a basal medium sufficient to support the serum-free growth of CHO cells and a supplemental medium, wherein the combined basal medium and supplemental medium include an antioxidant, L-glutamine, iron, ethanolamine, recombinant albumin, and recombinant insulin, in an amount sufficient to support the growth of a single CHO cell.

A serum-free CHO cell culture medium comprising a basal medium sufficient to support the serum-free growth of CHO cells and a supplemental medium, wherein the combined basal medium and supplemental medium include an antioxidant, L-glutamine, iron, ethanolamine, recombinant albumin, and recombinant insulin, and, optionally, one or more growth factors selected from the group consisting of recombinant epidermal growth factor and recombinant insulin like growth factor, in an amount sufficient to support the growth of a single CHO cell.

In particular embodiments of the present invention, the basal medium has substantially the same ingredients as a basal medium selected from the group consisting of VM-Soy and 50/50. The antioxidant is present in a concentration of from about 0.01 mM to about 10 mM, or more preferably from about 1 mM to about 3 mM. The L-glutamine is present in a concentration of from about 2 mM to about 6 mM, or more preferably from about 2 mM to about 3 mM. The iron is present in a concentration of from about 10 mg/L to about 400 mg/L, or more preferably from about 100 mg/L to about 300 mg/L. The ethanolamine is present in a concentration of from about 1 mg/L to about 100 mg/L, or more preferably from about 20 mg/L to about 40 mg/L. The recombinant albumin is present in a concentration of from about 1 g/L to about 5 g/L, or more preferably from about 1 g/L to about 3 g/L. The recombinant insulin is present in a concentration of from about 10 g/L to about 20 g/L, or more preferably from about 13 g/L to about 17 g/L. The recombinant EGF, if present, is present in a concentration of from about 0.01 μg/L to about 100 μg/L, or more preferably from about 5 μg/L to about 20 μg/L. The recombinant IGF, if present, is present in a concentration of from about 1 μg/L to about 1500 μg/L, or more preferably from about 50 μg/L to about 200 μg/L.

In a more particular embodiment of the present invention, the antioxidant is present in a concentration substantially equivalent to a 1× solution of Sigma Cat. No. A-1345, the L-glutamine is present in a concentration of about 2 mM, the iron is present in concentration of about 200 mg/L, the ethanolamine is present in a concentration of about 30 mg/L, the recombinant albumin is present in a concentration of about 2 g/L, and the recombinant insulin is present in a concentration of about 15 mg/L, the recombinant EGF, if present, is present in a concentration of about 10 μg/L, and the recombinant IGF, if present, is present in a concentration of about 100 μg/L.

In yet another aspect, the present invention is directed to a CHO cell in a culture media as described above.

As used herein, the term “cell culture” means the maintenance, growth, propagation, or expansion of cells in an artificial in vitro environment outside of a multicellular organism or tissue. Typically, cell culture is performed under sterile, controlled temperature and atmospheric conditions in tissue culture plates (e.g., 10-cm plates, 96-well plates, etc.), or other adherent culture (e.g., on microcarrier beads) or in suspension culture such as in roller bottles. Cultures can be grown in shake flasks, small scale bioreactors, and/or large-scale bioreactors. A bioreactor is a device used to culture cells in which environmental conditions such as temperature, atmosphere, agitation, and/or pH can be monitored, adjusted and controlled. A number of companies (e.g., ABS Inc., Wilmington, Del.; Cell Trends, Inc., Middletown, Md.) as well as university and/or government-sponsored organizations (e.g., The Cell Culture Center, Minneapolis, Minn.) offer cell culture services on a contract basis. Optimal periods for which the cultures are in contact with agents that select for the selectable activity are for longer than the typical period for one normal growth cycle (e.g., for Chinese hamster ovary cells (CHO cells), where one growth cycle has been reported to be approximately 20-22 hours (Rasmussen et al. (1998), Cytotechnology, 28:31-42)). As such, in one embodiment, the cultures comprise selectable conditions, e.g., drugs, metabolites, or color substrates, preferably for at least about one day, more preferably for at least about 3 days, and even more preferably for at least about 7 days.

The terms “cell culture medium,” “culture medium” and “medium formulation” refer to a nutritive solution for culturing or growing cells. The terms “media” (plural) and “medium” (singular) are synonymous and are used interchangeably herein, and use of one form of the term does not imply exclusion of the other form. The terms “cultivating,” “culturing,” “growing,” “maintaining,” “supporting,” and “expanding” are synonymous in meaning that cells remain viable and capable of producing progeny.

The term “ingredient” refers to any compound, whether of chemical or biological origin, that can be used in cell culture media to maintain or promote the growth or proliferation of cells. The terms “component,” “nutrient” and “ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain growth of cells ex vivo can be selected by those of skill in the art, in accordance with the particular need.

Particular methods of cell culture used in the present invention may include culture in various types of containers, including culture vessels, jars, bottles, vials, straws, ampules, and cryotubes.

The media of the present invention is used to effect appropriate culture of cells by bringing the media or its components into contact with all or part of the cell population. The media may be brought into contact with the cells by mixing, adding, combining, seeding, or stirring of one or more cells with one or more compounds, solutions, media, etc. Media may also be brought into contact with the cells all at once, incrementally, or in a step-wise manner by, for example, “feeding” or “fluid-changing” to replace or supplement the medium in which cells are cultured.

In a particular aspect, the present invention relates to a method and media for culturing very low density populations of CHO cells in a medium that is sufficient to support the growth of a single CHO cell. The phrase “sufficient to support the growth of a single cell” means that the media is capable of supporting growth of a single cell, but does not require that the media actually be used to support growth of a single cell. The media of the present invention is contacted with a population of CHO cells or their progeny, in growth conditions capable of sustaining clonal growth, that is, growth at very low cell densities, such as densities less than about 100 cells/ml, including single CHO cells. The process may be used either in the process of obtaining a clonal population of CHO cells (i.e., reducing the cell density to a single cell) or expanding a population of CHO cells from a single cell (i.e., increasing the cell density from a single cell). The CHO cells used in the present invention may therefore be precursors to or progeny derived from a single cloned progenitor cell. As will be appreciated by those in the art, conditions for growing cells in colonies is not equivalent to clonal growth conditions, which requires conditions sufficient to maintain viability and growth of cells derived from a single cell. It will be understood that the present invention is not limited to, although it encompasses, methods and media where a single CHO cell is being cultured. Methods and media sufficient to support the growth of a single CHO cell will, of course, be capable of supporting the growth of CHO cells at very low density cell conditions of, for example, 10 cells/ml, 10² cells/ml, 10³ cells/ml and 10⁴ cells/ml. The claimed invention thus contemplates that the methods and media of the invention include cell culture media that may also be used to culture CHO cells at a higher density than a single cell in a processes used to eventually obtain a single CHO cell, or expanding a single CHO cell to a larger population. Similarly, provided the culture media is capable of maintaining the growth a CHO cell, using such media to culture low density CHO cell populations is also encompassed in the present invention, even if it is not used in a process to actually obtain a single CHO cell or to expand a single CHO cell to a larger population. Whether a modified medium is sufficient to support the growth of a single CHO cell can be determined using the plating efficiency assay described below in Example 1.

In a particular aspect of the invention, the media comprises the following ingredients, in an amount sufficient to support the growth of a single CHO cell: a serum-free basal medium sufficient to support the serum-free growth of CHO cells at cell densities greater than about 10⁴ cells/ml and a serum-free supplemental medium providing one or more of the following ingredients, selected from the group consisting of an antioxidant, L-glutamine, an iron supplement, ethanolamine, albumin, and insulin, and wherein the final medium includes each of the ingredients (antioxidant, L-glutamine, an iron supplement, ethanolamine, albumin, and insulin), which may be included with either or both of the basal medium or supplemental medium. In another aspect of the invention, the ingredients of the supplemental medium consist essentially of an antioxidant, L-glutamine, an iron supplement, ethanolamine, albumin, and insulin, with the proviso that any one of the above ingredients may be provided in suitable quantities in the basal medium, and therefore be omitted from the supplemental medium, as long as the final medium contains these ingredients in quantities sufficient to support the growth of a single CHO cell.

In another aspect of the invention, the media comprises the following ingredients in an amount sufficient to support the growth of a single CHO cell: a serum-free basal medium sufficient to support the serum-free growth of CHO cells at cell densities greater than about 10⁴ cells/ml, and a serum-free supplemental medium providing one or more of the following ingredients, selected from the group consisting of an antioxidant, L-glutamine, an iron supplement, ethanolamine, albumin, insulin, and one or more growth factors selected from the group consisting of EGF and IGF and wherein the final medium includes each of the ingredients (antioxidant, L-glutamine, an iron supplement, ethanolamine, albumin, insulin, one or more growth factors selected from the group consisting of EGF and IGF), which may be included with either or both of the basal medium or supplemental medium. In another aspect of the invention, the ingredients of the supplemental medium consist essentially of an antioxidant, L-glutamine, an iron supplement, ethanolamine, albumin, insulin, and one or more growth factors selected from the group consisting of EGF and IGF, with the proviso that any one of the above ingredients may be provided in suitable quantities in the basal medium, and therefore be omitted from the supplemental medium, as long as the final medium contains these ingredients in quantities sufficient to support the growth of a single CHO cell.

As used above with respect to the definition of ingredients in the supplemental medium, the term “consisting essentially of” includes the listed ingredients, as well as additional ingredients that do not materially affect the sufficiency of the final medium to support the growth of a single CHO cell. It will be understood by those in the art that various additional ingredients may be added that do not materially affect the sufficiency of the final medium to support the growth of a single CHO cell. For example, additional ingredients may simply have no affect on the growth of cells, or may be present in a sufficient, low amount that it does not have a material affect on the growth of the cells at low density cell conditions. Thus, those in the art will also appreciate that some ingredients may materially affect the sufficiency of the final medium to support the growth of a single CHO cell at higher concentrations, while the same ingredient may not materially affect the sufficiency of the final medium to support the growth of a single CHO cell at lower concentrations. The term “consisting essentially of” therefore contemplates that the cell culture medium of the present invention may include in addition to the listed ingredients, additional ingredients that do not materially affect the sufficiency of the final medium to support the growth of a single CHO cell at low concentrations, even though such ingredients may materially affect the sufficiency of the final medium to support the growth of a single CHO cell at higher concentrations. Whether an additional ingredient materially affects the sufficiency of the final medium to support the growth of a single CHO cell can be determined using the plating efficiency assay described below in Example 1.

Any one of a number of methods of cloning known in the art are suitable for cloning a cell line, including diluting the progenitor cells to one cell, or less, per cell culture plate well, a method termed limiting dilution. Similarly, progenitor cells may be cloned with the use of cloning rings, by selective ablation, by dilute culture on microparticles, by single-cell sorting using flow cytometry, by picking individual cells with micropipet or optical tweezers, and by agar. Other methods include cloning by affinity-based interactions, e.g. affinity panning, immunosurgery in combination with complement, by flow cytometry, by centrifugal elutriation, by differential centrifugation, etc. It is understood, that numerous such methods of isolating CHO cells are known and acceptable to those in the art, and may be used in conjunction with the media and methods of the present invention. The present invention particularly relates to a method for culturing Chinese Hamster Ovary (CHO) cells. The term “CHO cell” means any of a wide variety of CHO cell lines suitable for growth in culture. CHO cell lines are available from, for example, the American Type Culture Collection (ATCC, Manassas, Va.) and NRRL (Peoria, Ill.). Some of the more established CHO cell lines typically used in the industrial laboratory include CS9 and AM-1/D cells (described in U.S. Pat. No. 6,210,924). In certain embodiments, cell lines may be selected by determining which cell lines have high expression levels and produce recombinant proteins with constitutive biological properties. Suitable CHO cell lines for use in the present invention include CHO-KI (ATCC# CCL-61), EM9 (ATCC# CRL-1861), UV20 (ATCC# CRL-1862), DUX B11 (Lawrence Chasin, University Columbia), CHO (ECACC# 85050302), CHO dfhr- (ECACC# 94060607), RR CHO KI (ECACC# 92052129), CS9 (derived from DUX B11 through adaptation to serum-free medium and sub cloning), and AM1/D (derived from DUX B11 through adaptation to serum-free medium and sub cloning).

The method of clonal isolation used will, in preferred aspects of the invention, be amenable to selective isolation of genetically engineered CHO cells that retain or demonstrate improved capacity to express some or all of a recombinant polynucleotide molecule. The term “genetically engineered” means that the cell line has been transfected, transformed or transduced with a recombinant polynucleotide molecule, so as to cause the cell to express a desired protein. The cell lines used in the present invention can be genetically engineered to express a protein of commercial or scientific interest. Methods and vectors for genetically engineering cells and/or cell lines to express a protein of interest are well known to those of skill in the art; for example, various techniques are illustrated in Current Protocols in Molecular Biology, Ausubel et al., eds. (Wiley & Sons, New York, 1988, and quarterly updates) and Sambrook et al., Molecular Cloning: A Laboratory Manual (Cold Spring Laboratory Press, 1989). Polynucleotide sequences encoding a particular protein are inserted into a suitable mammalian host cell by transformation of the host cell with the polynucleotide sequences. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,912,040, 4,740,461, and 4,959,455 (which patents are hereby incorporated herein by reference for any purpose). In certain embodiments, the transformation procedure used may depend upon the host to be transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

Following transformation of a suitable mammalian host cell with polynucleotide sequences encoding a recombinant protein, cells demonstrating stable expression of the recombinant protein are identified and isolated. Stable expression of a recombinant protein is achieved by transfection of appropriate DNA vectors into dihydrofolate reductase deficient (DHFR⁻) Chinese hamster ovary cells (CHO AM-1/D, U.S. Pat. No. 6,210,924) followed by isolation and testing of individual clones demonstrating highest expression of recombinant protein, in accordance with methods known in the art. Based on growth and production in small-scale spinners and larger scale bioreactors, a specific cell line is chosen as the cell line for manufacturing of the recombinant protein.

Cells producing the highest levels of recombinant protein may be cloned by methods well-known in the art, for example, by multiple rounds of limiting dilution in 96 and/or 24 well plates under serum-free conditions, using the cell culture media of the present invention. The clones are selected based on production and growth characteristics in various suspension vessels. Enzyme Immunoassays (EIAs) may be performed to select the clone that produces the highest level of recombinant protein. Growth characteristics, including doubling times and densities are then measured by growing the clones various shaker or spinner flasks and bioreactors ranging from 100 ml to up to 3 L. The clone with the fastest doubling time that reaches the highest density in culture is selected, and is selected as the cell line for use in commercial production.

The cell culture media used in the methods of the present invention may be solid or liquid, preferably liquid. Suitable synthetic media may include water, an osmolality regulator, a buffer, an energy source, amino acids including L-glutamine, an inorganic or recombinant iron source and a recombinant or synthetic growth factor and optionally non-ferrous metal ions, vitamins and cofactors.

The components of the medium are primarily inorganic, synthetic or recombinant. Some components may be obtained from a plant, fungal, yeast or bacterial source, provided such sources are not potential sources of infectious pathogens capable of causing disease. Recombinant components are prepared under highly pure conditions to minimize the risk of contamination from the parent tissue passing to the cells used to produce the components. Further purification steps may be employed to remove cell proteins. The culture media used in the process of the invention preferably contains no components derived from an animal source.

The cell culture media of the present invention is serum-free. As used herein, the term “serum-free” medium means a medium that excludes serum of any type (e.g., fetal bovine serum (FBS), horse serum, goat serum, etc.). Both the basal cell media and the supplemental media are serum-free.

The term “physiologically acceptable,” as used herein in reference to the concentration or amount of a particular compound, means the concentration or amount that generally exists in a natural state in a mammalian tissue.

Basal Media

The first component of the cell culture media of the present invention is a serum-free basal cell media. The term “basal medium” means any medium which is capable of supporting growth of CHO cells at cell density conditions greater than about 10⁴ cells/ml, either alone or when supplemented the serum-free supplement of the present invention. The basal medium supplies standard inorganic salts, such as zinc, iron, magnesium, calcium and potassium, as well as trace elements, vitamins, an energy source, a buffer system, and essential amino acids. Basal media which can be used in the present invention include but are not limited to standard basal media used for the serum-free culture of CHO cells at normal cell densities greater than about 10⁴ cells/ml, such as cell densities of about 10⁵ cells/ml, or greater than about 10⁶ cells/ml. Suitable basal media are commercially available from various sources, and include, for example, Iscove's Modified Dulbecco's Medium, RPMI 1640, Dulbecco's Modified essential Medium (DMEM), Minimal Essential Medium-alpha (MEM-alpha), MCDB media, Ham's F12, as well as combinations of such media. Particular combinations found to be useful include, for example, basal medium composed of 50% DMEM/F12 and 50% MCDB302 (referred to herein as 50/50), and the basal medium VM-Soy, the composition of which is disclosed below. Other basal media capable of sustaining growth of CHO cells at normal (high) cell density conditions are also known to those in the art, and may be used as a basal medium in the present invention.

Generally, the basal medium will contain inorganic salts for regulation of the osmolality of the cell cloning medium, preferably in the range 200-400 milli-Osmols (mOsm), and more preferably in the range 290-350 mOsm. Osmolality regulators are generally salts. Inorganic salts used in the basal medium of the present invention include, for example, salts of Ca²⁺, K⁺, Mg²⁻, Na⁺, CO₃²⁻, PO₄³⁻. Any salt of each of these moieties can be used to make the medium of the present invention. For example, the following salts can be used: CaCl₂, KCl, KNO₃, MgSO₄, NaCl, NaHCO₃, and NaH₂PO₄—H₂O.

The basal medium will also contain buffering agents, which act to stabilize the hydrogen ion concentration and therefore the pH of a solution by neutralizing, within limits, both acids and bases. Buffering agents commonly used in the cell cloning medium maintain the pH in the range 6.5-7.5, preferably about pH 7.0, and include carbonates such as NaHCO₃; chlorides, sulphates and phosphates such as CaCl₂.2H₂O, MgSO₄.7H₂O, NaH₂PO₄.2H₂O, Na₂HPO₄.7H₂O, beta-glycerol-phosphate, bicarbonate or sodium pyruvate. Such buffers are generally present in an amount 50-3000 mg/liter. Other buffers, such as N-[2-hydroxyethyl]piperazine-N′-[2-ethanesul-phonic acid] otherwise known as HEPES and 3-[N-Morpholino]-propanesul-fonic acid otherwise known as MOPS are generally present in an amount 1000-10,000 mg/liter. Preferably, a combination of bicarbonate and HEPES is used in the basal medium of the present invention.

The basal medium will further contain an energy source, such as a carbohydrate. The energy source of use in the cell cloning medium is preferably a monosaccharide, such as manose, fructose, galactose or maltose, preferably glucose, and particularly D-glucose, which is generally present in an amount 1000-10,000 mg/liter.

A basal medium will also contain various trace elements, which will be present in a cell culture medium in only trace amounts. Trace elements include, for example, Se⁴⁺, Ag⁺, Al⁺, Ba²⁺, Cd²⁺, Co²⁺, Cr⁺, Ge⁴⁺, Br⁻, I⁻, Mn²⁺, F⁻, Si⁴⁺, V⁵⁺, Mo⁶⁺, Ni²⁺, Rb⁺, Sn²⁺ and Zr⁴⁺ and salts thereof. Particularly common trace elements include non-ferous metal ions of magnesium, copper and zinc; as well as sodium, potassium and selenium. The ions are generally added to the medium in the form of salts such as chlorides and sulphates. Any salt of a given trace element can be used to make the basal medium of the present invention. For example, the sodium salt of selenium oxide (sodium selenite, Na₂SeO₃) is preferably used to provide selenium. Suitable concentrations of trace element-containing compounds can be determined by one of ordinary skill in the art. For example, in the medium of the invention, the concentration of SeO₃²⁻ can be about 0.007 to about 0.07 mg/L. In a preferred embodiment of the medium of the invention, the concentration of SeO₃²⁻ is about 0.01 mg/L. The amounts are typically similar to those provided in the media disclosed in the examples below, but may be varied.

Vitamins and enzyme co-factor vitamins (co-factors), such as Vitamin B6 (pyridoxine), Vitamin B12 (cyanocobalamin) and Vitamin K (biotin) may be present in the basal medium, preferably in an amount 0.01-0.5 mg/liter; Vitamin C (ascorbic acid) may be present in an amount 10-30 mg/liter, Vitamin B2 (riboflavin) may be present in an amount 0.1-1.0 mg/liter. The basal medium may also contain Vitamin B1 (thiamine), nicotine amide, Vitamin B5 (D calcium pentothenate), folic acid, i-inositol, which are preferably present in an amount of 0.2-8.0 mg/liter.

A basal medium will also typically include a lipid agent, which provide a source of lipids or contribute to lipid formation. Suitable lipid agents which can be used in the supplement and the medium of the present invention include, but are not limited to, Human Ex-Cyte® (Bayer), ethanolamine (or a salt thereof), sitosterol (a plant steroid), rice hydrolysate (a mixture of proteins and lipids), LTI Defined Lipid Mixture, a mixture of arachidonic acid, cholesterol, DL-alpha-tocopherol-acetate, ethyl alcohol, linoleic acid, linolenic acid, myristic acid, oleic acid, palmitric acid, palmitic acid, Pluronic® F-68, stearic acid, and Tween® 80, choline chloride, phosphatidylcholine or methyl lineoleate. Preferably, ethanolamine is used in the supplement and the medium of the invention. Suitable concentrations of a lipid agent ingredient can be determined by one of ordinary skill in the art. Lipid agents will generally be present in an amount of 0.05-10 mg/liter.

Additional amino acids (preferred forms in parentheses), listed in Table 2, may also be added to the basal medium, in the indicated preferred concentrations.

TABLE 1

| Amino Acid | Preferred mg/liter |
|---|---|
| L-Alanine | 20-50 |
| L-Arginine (HCl) | 50-100 |
| L-Asparagine (H₂O) | 20-50 |
| L-Aspartic Acid | 20-50 |
| L-Cystine (disodium salt) | 50-100 |
| L-Glutamic acid | 50-100 |
| L-Glutamine | 400-600 |
| Glycine | 20-50 |
| L-Histidine (HCl.H₂O) | 30-60 |
| L-Isoleucine | 50-150 |
| L-Leucine | 50-150 |
| L-Lysine (HCl) | 100-200 |
| L-Methionine | 20-50 |
| L-Phenylalanine | 40-80 |
| L-Proline | 30-60 |
| L-Serine | 30-60 |
| L-Threonine | 50-120 |
| L-Tryptophan | 10-20 |
| L-Tyrosine (disodium salt) | 50-120 |
| L-Valine | 80-120 |

The amino acids included in the basal medium are preferably of synthetic origin. The amounts which are usually included vary for each amino acid but are generally in the range 10-150 mg/ml. However, L-glutamine is generally present at much higher concentration preferably in the range 400-600 mg/ml.

A pH indicator may be advantageously included in the basal medium, for example, Phenol red sodium salt, at a concentration of about 5-50 mg/liter.

The basal medium may also contain selenium (optionally in the form of sodium selenite), generally in an amount 0.01-0.2 mg/liter, or L-Ascorbic acid, generally in an amount 20-50 mg/liter, to help minimize the potential toxic effects of ferrous or ferric ions, and oxygen. Further use of chelating agents such as citrate or Ethylenediaminetetraacetic acid (EDTA) or a free radical scavenger such as alpha-Tocepherol (vitamin E) are advantageous in reducing free radical damage.

It is also preferable to add to the medium, a compound such as putrescine, advantageously as a salt such as HCl, which is known to play a role in maintaining the structure of the endoplasmic reticulum and to be required by certain CHO cell lines to support growth. Putrescine or a salt thereof is preferably added in an amount 0.01-1.0 mg/liter.

Serum-free media may also contain hypoxanthine or thymidine. In preferred embodiments, the serum-free media is essentially free of hypoxanthine or thymidine, which may bypass the selection pressure placed on the dhfr selection and amplification system as previously disclosed. The result may be loss of genetic material specifying the product and the dhfr genes.

The basal culture medium of the present invention supports CHO cell growth and when supplemented with an appropriate agent such as methotrexate for the dhfr system usually in an amount 0.05-5.0 μM, (or MSX for the GS system), allow full selection pressure to be exerted on the cells. It will be understood that hypoxanthine and thymidine at concentrations which are insufficient to bypass selection of the dhfr system may be present in the medium, but the presence of these two nucleotide precursors is not preferred for use with the present invention.

In large scale bioreactors, CHO cells are particularly susceptible to sheer forces arising from the sparging of the vessel with gases and the mixing with the impeller. To minimize the occurrence of cellular damage it is advantageous for the medium to contain a cell protectant such as polyethylene glycol, polyvinyl alcohols or pluronic polyols. Of these, Pluronic® (polyol, BASF Wyandotte Corp.) polyol F68 is preferred since unlike polyvinyl alcohols this is a non-toxic substance and unlike polyethylene glycols does not interfere with downstream purification.

The CHO cell basal medium also may supplemented with a peptide digest, hydrolysates or extracts, such as Tryprone, casein hydrolysate, yeast extract, or preferably papain digested soy peptone. As demonstrated in the examples below, the addition of hydrolysates (such as Hy Soy) at high concentrations or supplementation beyond the normal basal medium level, may inhibit growth of CHO cells at low cell densities. Accordingly, such hydrolysates should not be present in the cell culture medium of the invention at concentrations more than about 6000 mg/L. Preferably, hydrolysates will be present only in the basal medium, and no additional amounts added in the supplemental medium. However, in the event that lower amounts of hydrolysates are included in the basal medium, limited additional amounts may be added to the supplemental medium without materially altering the ability of the supplemental medium to support the growth of single CHO cells or CHO cells at low density conditions.

Other growth factors which may be added to the basal medium include growth factors such as platelet-derived growth factor (PDGF), thyroxtne T₃, thrombin, interleukins such as IL2 and IL6, progesterone, hydrocortisone and vitamin E. Folic acid, vitamin B6 and vitamin B12 which are involved in the folate pathway may also be added to enhance the growth of cells. Polypeptide compounds are preferably of synthetic or recombinant origin.

The cell cloning process of the present invention will typically begin with a high density cell culture containing a population of transformed CHO cells greater than about 10⁵ cells/ml, cultured in a basal cell media. A typical basal cell media may contain, for example, Dulbecco's Modified Eagle's Medium/Ham's Nutrient F12 (DMEM/F12, 1:1) (Sigma-Aldrich, St Louis, Mo.) or MCDB302 (Sigma-Aldrich, St Louis, Mo.). In particular embodiments, the basal cell culture medium for use throughout the entire cell culture process may be a medium comprising 50% DMEM/F12 and 50% MCDB302. Alternatively, a suitable basal cell culture media may be VM-Soy media, which is comprised of 50% DMEM/F12, 50% MCDB302, plus other supplemental levels of amino acids, additional nutrients and salts, a soy hydrolysate and recombinant human insulin, as described in U.S. Patent Publication No. 2004/0033535 A1, the contents of which are incorporated herein by reference. The contents of VM-Soy media are set forth in Table 2 as follows:

TABLE 2
VM-Soy Cell Culture Media Components for Basal Media

| DMEM/F12 | VMSoy Batch |
|---|---|
| COMPONENTS | Medium (mg/L) |
| Inorganic salts | |
| CaCl₂ (anhyd.) | 116.60 |
| CuSO₄.5H₂O | 0.0026 |
| Fe(NO₃)₃.9H₂O | 0.1000 |
| FeSO₄.7H₂O | 0.8340 |
| KCl | 311.80 |
| MgCl₂ (anhyd.) | 57.280 |
| MgSO₄ (anhyd.) | 97.680 |
| NaCl | 905.990 |
| NaH₂PO₄.H₂O | 125.00 |
| Na₂HPO₄ | 142.040 |
| ZnSO₄.7H₂O | 0.8640 |
| Other Components | |
| D-Glucose | 3151.00 |
| Na Hypoxanthine | 5.40 |
| Linoleic acid | 0.090 |
| Lipoic acid | 0.2060 |
| Phenol Red | 8.10 |
| Putrescine.2HCl | 0.1620 |
| Sodium Pyruvate | 110.00 |
| Amino acids | |
| L-Alanine | 26.70 |
| L-Arginine HCl | 295.00 |
| L-Asparagine.H₂O | 45.00 |
| L-Aspartic acid | 39.90 |
| L-Cysteine.HCl.H₂O | 35.120 |
| L-Cystine.2HCl | 62.580 |
| L-Glutamic acid | 44.10 |
| L-Glutamine | 657.00 |
| Glycine | 52.50 |
| L-Histidine.HCl.H₂O | 62.950 |
| L-Isoleucine | 108.940 |
| L-Leucine | 118.10 |
| L-Lysine HCl | 182.50 |
| L-Methionine | 34.480 |
| L-Phenylalanine | 70.960 |
| L-Proline | 57.50 |
| L-Serine | 73.50 |
| L-Threonine | 106.90 |
| L-Tryptophan | 18.040 |
| L-Tyrosine.2Na.2H₂O | 111.580 |
| L-Valine | 105.70 |
| Vitamins | |
| Biotin | 0.0073 |
| D-Ca Pantothenate | 4.480 |
| Choline Chloride | 17.960 |
| Folic Acid | 5.30 |
| i-Inositol | 25.20 |
| Niacinamide | 4.040 |
| Pyridoxal HCl | 4.00 |
| Pyridoxine HCl | 0.0620 |
| Riboflavin | 0.43 80 |
| Thiamine HCl | 4.340 |
| Thymidine | 0.3635 |
| Vitamin B12 | 1.360 |
| ADDITIONAL COMPONENTS | |
| Rhu insulin | 5.00 |
| Selenous Acid | 0.0050 |
| Ethanolamine | 0.0012 |
| Triiodothyronine | 0.000040 |
| Hydrocortisone | 0.020 |
| Ferric Citrate | 122.450 |
| Pluronic F-68 | 1000.00 |
| Soy Hydrolysate | 6000.00 |
| NaHCO₃ | 3000.00 |
| NaCl | 3500.00 |

The basal cell media used for initial culturing of recombinant CHO cells are capable of supporting the growth and secretion of product from such cells in suspension in small and large scale bioreactors, static cultures and/or spinners, at high cell density namely greater than about 1×10⁵ cells/ml up to or greater than about 1.5×10⁶ cells/ml and product secretion of 30 mg/L up to greater than about 150 mg/L. The basal medium used in the invention is also capable of supporting this growth and product secretion over multiple passages lasting up to or greater than 6 months.

Supplemental Media

The cell culture media of the present invention also comprises a “supplemental medium,” which may be added to the serum-free basal cell media at a stage of the cloning process when very low cell density conditions are reached, for example, when the cell density is at or below 10⁵ cells/ml, 10⁴ cells/ml, 10³ cells/ml, 10² cells/ml, or 10 cells/ml, or at a single cell. In a particular embodiment of the present invention, individual CHO cells producing the highest levels of recombinant protein are cloned by multiple rounds of limiting dilution in 96- and/or 24-well plates under serum-free conditions. When cell colonies are diluted to low density conditions (i.e., less than about 1×10⁴ cells/ml), the basal cell media described above is supplemented with the supplemental cell culture medium. In another aspect of the invention, a defined medium comprising the basal medium and the supplemental medium in combination may be used to culture a low density population of CHO cells that has been isolated in by means other than gradual reduction of the cell density, for example, by physical selection of a single cell from a high density population of cell. In this case, the single cell may be combined with the complete medium of the present invention, comprising both the basal medium and the supplemental medium.

In particular embodiments, the supplemental medium used in accordance with the present invention consists essentially of antioxidants, ethanolamine, Epidermal Growth Factor (EGF), iron supplements, L-glutamine, insulin and albumin. In an alternative embodiment, the supplemental medium used in accordance with the present invention consists essentially of antioxidants, ethanolamine, Epidermal Growth Factor (EGF), iron supplements, L-glutamine, insulin, albumin, and recombinant Insulin Like Growth Factor (Long R3 IGF-1).

In another embodiment, the combined basal medium and supplemental medium may comprise the standard ingredients of a basal medium, as described above, with elevated concentrations of the following supplemental ingredients: antioxidants, ethanolamine, Epidermal Growth Factor (EGF), iron supplements, L-glutamine, insulin, albumin, and, optionally, Insulin Like Growth Factor (Long R3 IGF-1). It will be understood by those in the art that the ingredients of the supplemental medium may be added to the basal medium and omitted from the supplemental medium, so long as the ingredients are present in the final combined medium.

The ingredients of the supplemental medium are described in the following paragraphs:

Antioxidants

The supplemental medium of the present invention includes an antioxidant or combination of antioxidants. Antioxidants protect hematopoietic cells from the detrimental (and often lethal) effects of multiple free radical species and other toxic moieties known to be generated in the cell culture environment. Thus, by addition of antioxidants according to the invention, shelf-life of a medium can be increased or enhanced. Moreover, to the extent that such detrimental products are formed during the storage of a medium, addition of antioxidants according to the invention provide for regeneration of the medium, thereby enhancing or increasing the capacity or the ability of the medium to expand cells. The addition of antioxidants to achieve such beneficial results can be applied to any cell culture media, particularly serum-free media.

The term “antioxidant” means molecules, which inhibit reactions that are promoted by oxygen or peroxides. Antioxidants that may be used in the supplement or the medium of the present invention include but are not limited to N-acetyl-L-cysteine or derivatives thereof (see International Patent Application WO 95/00136), 2-mercaptoethanol or derivatives thereof, D,L-tocopherol acetate or derivatives thereof, ascorbic acid or derivatives thereof, thiol compounds, such as dithiothreitol and glutathione, or derivatives of thiol compounds, catalase or derivatives thereof, cysteine or derivatives thereof, thiolactate or derivatives thereof, penicillamine or derivatives thereof, mercaptoethanesulfonic acid or derivatives thereof, and mercaptopropionic acid, or derivatives thereof. Preferably, the antioxidants used in the supplement and the medium of the present invention are N-acetyl-L-cysteine, 2-mercaptoethanol, and D,L-tocopherol acetate or mixtures or derivatives thereof. In a preferred aspect of the present invention, the antioxidants are obtained from a commercially available source, such as Sigma-Aldrich (Antioxidant Supplement, Cat. No. A-1345), which in a preferred aspect of the invention is present in the final cell culture medium at a IX concentration (about 1 ml/L of a 1000× stock solution).

In accordance with the present invention, antioxidants are present in the supplemental medium in a concentration sufficient to support the growth of a single CHO cell. For example, antioxidants may be present in the supplemental medium in a concentration of from about 0.01 mM to about 10.0 mM, more preferably in a concentration of from about 0.1 mM to about 1.0 mM, and most preferably in a concentration of about 0.5 mM

Ethanolamine

The supplemental medium of the present invention also includes ethanolamine, or a salt thereof. Ethanolamine is a precursor for lipid synthesis, and supports growth of cells by providing the necessary precursor materials for synthesis of cellular membranes. Ethanolamine is commercially available from various sources, including Sigma-Aldrich (Cat. No. E-0135).

In a particular aspect of the invention, the supplemental medium includes ethanolamine in a concentration sufficient to support the growth of a single CHO cell. For example, ethanolamine may be present in the supplemental medium in a concentration of from about 0.01 mg/L to about 200 mg/L, more preferably in a concentration of from about 0.1 mg/L to about 100 mg/L, and most preferably in a concentration of about 30 mg/L.

Iron Supplement

The supplemental medium of the present invention also includes an iron source. As used herein, the term “iron” means a non-animal derived source of iron used to supplement the medium. The iron source is preferably inorganic, and includes, for example, ferric and ferrous salts such as ferric citrate or ferrous sulphate. The chelated salts such as ferric citrate and ferric ammonium citrate are preferred. However, other iron sources may be used which is not isolated from an animal source, for example, chemical iron chelators or recombinant protein iron carriers that provide equivalent amounts of iron. Iron chelate compounds which may be used include but are not limited to iron chelates of ethylenediaminetetraacetic acid (EDTA), ethylene glycol-bis(.beta.-aminoethyl ether)-N,N,N′,N′-tetraacetic acid (EGTA), deferoxamine mesylate, dimercaptopropanol, diethylenetriamine-pentaacetic acid (DPTA), and trans-1,2-diaminocyclohexane-N,N,N′,N′-tetraacetic acid (CDTA), as well as a ferric citrate chelate and a ferrous sulfate chelate. A particularly preferred source of iron is an iron supplement that is available from Sigma-Aldrich (Iron Supplement, Cat. No. 1-3153), which is preferably present in the final volume of the supplemental medium in a concentration of 0.2-2 ml/L, and more preferably about 1 ml/L, of the commercially available 1000× stock solution. Another acceptable source of iron is a product available from Biosource (Stock #4 100×, Catalog No. 431-020), which may be added to the medium of the present invention at a final cell culture concentration of 0.42 mg/L of ferrous sulfate heptahydrate.

The concentration of ferric or ferrous ions should be carefully controlled as these may help generate superoxides and free radicals in the medium, which may damage not only the cells themselves, but medium components and the desired end product. In a particular aspect of the invention, the supplemental medium includes iron supplements in a concentration sufficient to support the growth of a single CHO cell. For example, iron supplemental may be present in the supplemental medium in a concentration of from about 10 mg/L to about 400 mg/L, more preferably in a concentration of from about 10 mg/L to about 200 mg/L, and most preferably in a concentration of about 25-60 mg/liter.

The ingredients of the culture medium may be added in any order but it is preferable to add the iron source and when used, tyrosine, last to avoid precipitation.

L-Glutamine

The supplemental medium of the present invention also includes L-glutamine. L-glutamine is a required nutrient in the present invention. Many stock basal medias are provided with L-glutamine included. However, because L-glutamine will degrade by oxidation, such that all L-glutamine will be degraded over a period of a few weeks following manufacture, additional L-glutamine is typically included in a supplemental medium, which is added to the basal medium of the present invention as described above to compensate for degradation. Suitable sources of L-glutamine are available from various commercial sources, such as Gibco (Cat. No. 25030-081).

In a particular aspect of the invention, the supplemental medium includes L-glutamine in a concentration sufficient to support the growth of a single CHO cell. For example, L-glutamine may be present in the combined medium (basal medium plus supplemental medium) in a concentration of from about 2.0 mM to about 10.0 mM, more preferably in a concentration of from about 3.0 mM to about 5.0 mM, and most preferably in a concentration of about 4.0 mM. Thus, when the basal medium already contains about 2.0 mM L-glutamine, the supplemental medium will accordingly contain about an additional 2.0 mM L-glutamine.

Albumin

In accordance with the present invention, the serum-free supplemental medium includes albumin. Albumin is the most abundant protein contained in plasma. It is produced in the liver, and contributes to the maintenance of osmotic pressure in blood and binds to nutrients and metabolites to transport these substances. In cell culture media, albumin is frequently present as the dominant component of serum. However, the precise role of albumin in low density cell culture has not previously been understood or appreciated. There has been some speculation in the prior art that albumin should eliminated (see, e.g., U.S. Pat. No. 5,633,162) in high-density CHO cell culture media used for commercial production of recombinant proteins.

In a particular aspect, the present invention provides a biochemically defined serum-free culture medium containing albumin for use in cell cloning of engineered CHO cells. The presence of albumin has unexpectedly been shown to effectively maintain viability and growth of cells at low density conditions used throughout the cell cloning processes, and surprisingly is equivalent in cell cloning efficiency to media with serum.

As used herein, the term “albumin” means a polypeptide compound having the biological activity of albumin. Albumin may be any animal or mammalian albumin, such as human, bovine, equine, murine, porcine, Chinese hamster, chicken or rat, provided that the albumin is not directly derived from a natural animal source. Preferably, the albumin is human albumin. In the supplement and the medium of the present invention, the concentration of albumin, which facilitates cell growth, expansion, and differentiation in culture, can be determined using routine experimentation.

In a preferred aspect of the present invention, albumin is of synthetic or recombinant origin, and not derived from an animal source. The production of recombinant human albumin (rHA) is well known in the art. In one aspect of the invention, rHA is obtained from genetically modified yeast, which produce a human albumin protein. One such methodology for the production of rHA from yeast is taught in U.S. Pat. No. 5,612,197. Recombinant human albumin is available from various commercial sources, such as Sigma-Aldrich (Recombinant HSA, Cat. No. A-7223).

In accordance with the present invention, the final cell culture medium includes recombinant albumin in a concentration sufficient to support the growth of a single CHO cell. For example, recombinant albumin may be present in the final medium in a concentration of from about 1 g/L to about 10 g/L, more preferably in a concentration of from about 1 g/L to about 5 g/L, and most preferably in a concentration of about 2 g/L. In one aspect of the invention, the basal medium will not contain recombinant albumin, and the recombinant albumin is added in the above concentrations together with the supplemental medium when cell density conditions of less than about 10⁴ are reached. However, in the event that some recombinant albumin is included in the basal medium, the amount of albumin required in the supplemental medium may be less than the above amounts without materially altering the ability of the supplemental medium to support the growth of single CHO cells or CHO cells at low density conditions.

Insulin

The supplemental medium of the present invention also includes insulin. A number of insulins are known to those of ordinary skill in the art. See Gilman, A. G. et al., Eds., The Pharmacological Basis of Therapeutics, Pergamon Press, New York, 1990, pp. 1463-1495. Preferably, insulin, rather than an insulin substitute, is used in the supplement and the medium of the present invention. More preferably, the insulin is zinc insulin. Most preferably, the insulin is human zinc insulin. In the supplement and the medium of the present invention, the concentration of the insulin substitute which facilitates cell growth, expansion, and differentiation in culture can be determined using only routine experimentation. In a preferred aspect of the invention, the insulin is of synthetic or recombinant origin. Insulin is advantageously obtained by recombinant DNA techniques.

Insulin is preferably present in the supplemental medium in a concentration greater than about 5 mg/liter. In particular embodiments, insulin is present in the supplemental medium in a concentration of about 10 mg/liter. In another embodiment, insulin is present in the supplemental medium in a concentration of about 15 mg/L. In yet another embodiment, insulin is present in the supplemental medium in a concentration greater than about 20 mg/L.

In one aspect of the present invention, insulin may also be present in the basal media, in which case the concentration of insulin in the supplemental medium may be adjusted accordingly. In a particular aspect of the invention, the final media (containing both the basal medium and the supplemental medium) will preferably contain insulin in a concentration of about 10 mg/L. In another aspect, the final media will contain insulin in a concentration of about 15 mg/L. Optimally, where the basal medium already contains insulin in a concentration of about 5 mg/L, the supplemental medium will contain insulin in a concentration of about 10 mg/L, bringing the total insulin concentration to about 15 mg/L. In another aspect of the invention, the basal medium will contain recombinant insulin, and additional amounts of recombinant insulin are added with the supplemental medium when cell density conditions of less than about 10⁴ are reached. In the event that some recombinant insulin is included in the basal medium, the amount of insulin required at low cell density conditions may be less than the above amounts without materially altering the ability of the supplemental medium to support the growth of single CHO cells or CHO cells at low density conditions. In the event that the basal medium already contains about 15 mg/L of insulin, the supplemental medium may omit additional amounts of insulin.

EGF

In accordance with the present invention, the serum-free supplemental medium also includes epidermal growth factor (EGF). EGF may be any animal or mammalian EGF, such as human, bovine, equine, murine, porcine, Chinese hamster, chicken or rat, provided that the EGF is not directly derived from a natural animal source. Preferably, the EGF is human EGF. The EGF used in the presently claimed medium is preferably of synthetic or recombinant origin. EGF is available from various commercial sources, including Sigma-Aldrich (Cat. No. E-4269).

In a particular aspect of the invention, the supplemental medium includes EGF in a concentration sufficient to support the growth of a single CHO cell. For example, EGF may be present in the supplemental medium in a concentration of from about 0.1 μg/L to about 100 μg/L, more preferably in a concentration of from about 5 μg/L to about 20 μg/L, and most preferably in a concentration of about 10 μg/L.

IGF

In accordance with the present invention, the serum-free supplemental medium also includes insulin-like growth factor (IGF). IGF may be any animal or mammalian IGF, such as human, bovine, equine, murine, porcine, Chinese hamster, chicken or rat, provided that the IGF is not directly derived from a natural animal source. Preferably, the IGF is human IGF. The IGF used in the presently claimed medium is preferably of synthetic or recombinant origin. IGF is available from various commercial sources, including JRH (Cat. No. 85580).

IGF performs similar functions as insulin, and is not therefore an essential ingredient of the medium when adequate concentrations of insulin are present. In the event lower concentrations of insulin are used, IGF may appropriately be used to compensate for the lower insulin levels. Even with adequate levels of insulin, however, IGF may still be advantageously used. In a particular aspect of the invention, the supplemental medium includes IGF in a concentration sufficient to support the growth of a single CHO cell. For example, IGF may be present in the supplemental medium in a concentration of from about 10 μg/L to about 200 μg/L, more preferably in a concentration of from about 20 μg/L to about 150 μg/L, and most preferably in a concentration of about 100 μg/L. The amount of IGF used will vary, depending on whether IGF is present and the amounts of IGF present in the basal medium. In certain aspects of the invention, the final medium will contain from about 20 μg/L to about 150 μg/L of IGF. In preferred aspects of the invention, the final medium will contain about 100 μg/L of IGF.

Recombinant Proteins

In preferred embodiments of the present invention, the polypeptides used in the basal medium and supplemental medium (e.g., albumin, insulin, EGF, and IGF) will be of synthetic or recombinant origin.

Albumin and insulin, as used in accordance with the present invention, may be any form of mammalian albumin, including human, bovine, porcine, or Chinese Hamster. Preferably, the recombinant proteins are human.

The recombinant proteins used in the present invention are synthetic or recombinant, so as to avoid use of naturally purified sources that could contain residual contaminants. Recombinant proteins are preferably produced by recombinant expression in host cells. Such recombinant proteins are prepared under highly pure conditions to minimize the risk of contamination from the parent tissue passing to the cells used to produce the protein. The use of recombinant host cells to produce proteins is a highly cost effective method for obtaining large quantities of a protein for use in commercial applications. Recombinant proteins are typically expressed in a suitable host, for example, eukaryotic cells (such as yeast and animal cells), or procaryotic cells (such as E. coli or other type of bacteria), using a standard expression vector such as a plasmid, bacteriophage or naked DNA, and the protein expressed from the plasmid or DNA integrated into the host chromosome. Expression in eukaryotic systems is preferred, as such systems typically have the requisite cellular machinery to correctly fold complex mammalian proteins. Expression in prokaryotic systems typically results in superior yield, but requires further processing to assure correct folding of the protein. Recombinant human serum albumin is available from various commercial sources (e.g., Sigma Aldrich).

Medium Concentrations

The supplement or the medium of the present invention can be in liquid form or can be maintained in dry form. The type of liquid carrier and the method used to dissolve the ingredients into solution vary and can be determined by one of ordinary skill in the art with no more than routine experimentation. In general, the liquid carrier is water.

The present invention includes methods of growing CHO cells employing the medium described above. The medium may be added at the start of culture, or can be added in a fed-batch or in a continuous manner. Moreover, the components of the medium supplement may be added together, or separately, at different stages of the media production.

The supplement or the medium of the present invention can be made as a concentrated formulation (greater than 1× to 1000×) or as a 1×formulation. Preferably, the solutions comprising ingredients are more concentrated than the concentration of the same ingredients in a 1.times. media formulation. For example, the ingredients can be 10 fold more concentrated (10×formulation), fold more concentrated (25×formulation), 50 fold more concentrated (50×concentration), or 100 fold more concentrated (100×formulation). In particular, the supplement or the medium of the present invention can be made by dividing the ingredients into compatible, concentrated subgroups. See U.S. Pat. No. 5,474,931.

If the ingredients of the supplement or the medium are prepared as separate concentrated solutions, an appropriate (sufficient) amount of each concentrate is combined with a diluent to produce a less concentrated formulation or a 1×formulation. Typically, the diluent for the subgroups used is water but other solutions including aqueous buffers, aqueous saline solution, or other aqueous solutions may be used according to the invention.

Use in Recombinant Production of Proteins

The cell culture media of the present invention is preferred for the production of all types of proteins and antibodies, natural and recombinant. The invention therefore includes production of human antibodies wherein the amino acid sequences of the heavy and light chains are homologous with those sequences of antibodies produced by human lymphocytes in vivo or in vitro by hybridomas, or by transgenic non-human animals that are modified to produce human antibodies. Also provided are hybrid antibodies in which the heavy and light chains are homologous to a natural antibody but are combined in a way that would not occur naturally. For example, a bispecific antibody has antigen binding sites specific to more than one antigen. The constant region of the antibody may relate to one or other of the antigen binding regions or may be from a further antibody. Altered antibodies, for example chimeric antibodies have variable regions from one antibody and constant regions from another. Thus, chimeric antibodies may be species/species chimaeras or class/class chimaeras. Such chimeric antibodies may have one or more further modifications to improve antigen binding ability or to alter effector functioning. Humanized or CDR-grafted antibodies (EP 239400) are embraced within the invention, in particular Campath 1H (EP328404) (Campath is a trademark of The Wellcome Foundation) also composite antibodies, wherein parts of the hypervariable regions in addition to the CDRs are transferred to the human framework. Additional amino acids in the framework or constant regions of such antibodies may be altered. The invention further includes the production of Fab fragments which are roughly equivalent to the Y branch portions of the heavy and light chains; this includes incomplete fragments or fragments including part of the Fc region.

The serum-free media of the present invention has been shown to be effective in maintaining multiple recombinant cell lines expressing various recombinant proteins, including antibody molecules. Furthermore, the media has been used to successfully obtain a cell line for one recombinant protein that was derived through a completely serum-free process, including transfection, amplification, single cell cloning, and scale up. The expression and the growth rate of the various cell lines is not compromised by the elimination of serum derived nutrients.

### EXAMPLE 1

Formulations of culture media comprising albumin were tested to evaluate performance in culturing mammalian host cell lines at low density conditions. The growth performance of cells (CS9 and AM-1/D CHO host cells (DUXB11 derived lines) cultured in cloning media having formulations designated 1.0 and 1.1 (described in detail below in Table 3 and Table 4, respectively) were compared to a formulation with serum (positive control) and a formulation without serum (negative control). The positive control is a cell cloning culture media comprising 50% DMEM/F12 and 50% MCDB302, plus 3% dialyzed FBS, without the addition of other components. The negative control is the same 50/50 cloning media without FBS or other components. The 50/50 cloning medium is composed of 50% DMEM/F12 and 50% MCDB302. VM-Soy is a basal growth medium, described above.

CHO cells were cultured using serial limited dilution, the concentration of CHO cells was reduced to 1 cell/100 μL in basal growth media supplemented with the indicated supplemental formulation. 100 μL of the media was plated into 96 well plates. On days 10-14, the plates were screened for single cell colonies. The plates were then fed 25-50 μL of basal media combined with the supplemental formulation. Upon reaching confluence, the cells were then expanded into larger vessels containing a richer growth media.

TABLE 3
Formulation 1.0

| Ingredient | Final Volume |
|---|---|
| Antioxidant (Sigma: A-1345) | 1 ml/L of 1000 X |
| Ethanolamine (Sigma: E-0135) | 30 mg/L |
| Epidermal Growth Factor (EGF) (Sigma: E-4269) | 10 μg/L |
| Iron Supplement (Sigma: I-3153) | 1 ml/L of 1000 X |
| Insulin (Sigma: I-9278) | 10 mg/L |
| L-glutamine (Gibco: 25030-081) | 10 ml/L of 100 X |
| Albumin (Sigma: A-7223) | 2 g/L |
| Insulin Like Growth Factor (Long R3 IGF-1) | 100 μg/L |
| (JRH: 85580) | |

TABLE 4
Formulation 1.1

| Ingredient | Final Volume |
|---|---|
| Antioxidant (Sigma: A-1345) | 1 ml/L of 1000× |
| Ethanolamine (Sigma: E-0135) | 30 mg/L |
| Epidermal Growth Factor (EGF) (Sigma: E-4269) | 10 μg/L |
| Iron Supplement (Sigma: I-3153) | 1 ml/L of 1000× |
| Insulin (Sigma: I-9278) | 10 mg/L |
| L-glutamine (Gibco: 25030-081) | 10 ml/L of 100× |
| Albumin (Sigma: A-7223) | 2 g/L |

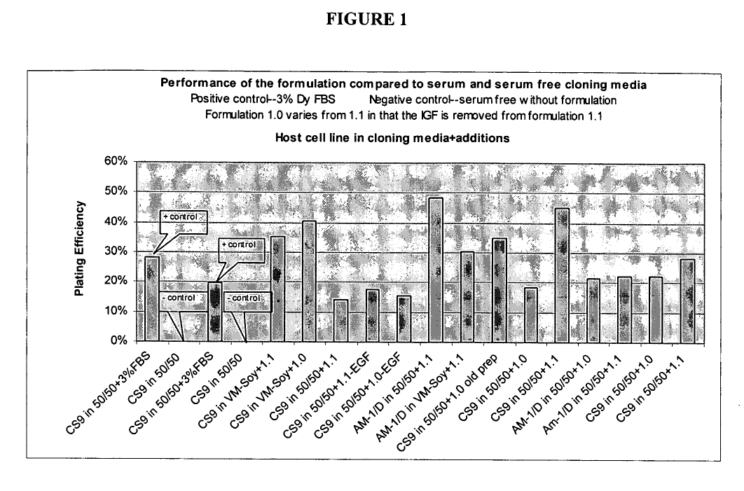

FIG. 1 shows the performance of various cell culture media on the growth of two different CHO cell lines, CS9 and AM-1/D. Performance is measured in terms of plating efficiency, which is the ratio of the number of wells in a 96-well-plate that successfully produced a viable colony to that of the total number of wells seeded at one cell per well. As seen in FIG. 1, the addition of either formulation 1.0 or formulation 1.1 to the cloning media supports the growth of the single cell equally in comparison to that of the cloning media containing serum. Moreover, with no additions to the 50/50 cloning media there is no growth or survival detected. Thus, the data shows that single cell growth requires either the addition of serum or one of the formulations 1.0 or 1.1 to survive and proliferate.

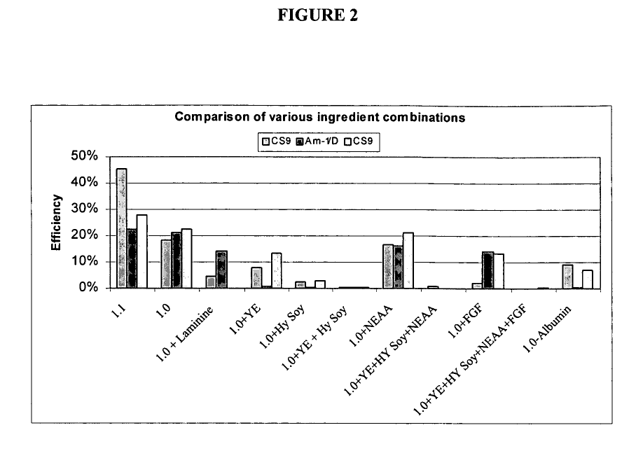

FIG. 2 shows the effects of the addition and/or deletion of various key components on the growth and survival of the single cell colonies. As shown in FIG. 2, efficiency of cell growth is dramatically reduced by the addition of certain components, and is optimum using formulation 1.0 or 1.1 alone.

Equivalents and References

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

What is claimed is:

1. A method of culturing a population of CHO cells at a cell density of less than about 100 cells/ml in a serum-free cell culture medium, comprising the steps of:
(a) culturing a population of CHO cells at a cell density greater than about 10⁴ cells/ml in a basal medium sufficient to support the serum-free growth of CHO cells;
(b) reducing the CHO cell density to less than about 100 cells/ml; and
(c) adding to the basal medium a supplemental medium, wherein the combined basal medium and supplemental medium include an antioxidant, L-glutamine, iron, ethanolamine, recombinant albumin, and recombinant insulin, in an amount sufficient to support the growth of a single CHO cell.

2. The method of claim 1, wherein the basal medium has substantially the same ingredients as a basal medium selected from the group consisting of VM-Soy and 50/50.

3. The method of claim 1, wherein the combined basal medium and supplemental medium include:
antioxidant in a concentration of from about 0.01 mM to about 10 mM;
L-glutamine in a concentration of from about 2 mM to about 6 mM;
iron in a concentration of from about 10 mg/L to about 400 mg/L;
ethanolamine in a concentration of from about 1 mg/L to about 100 mg/L;
recombinant albumin in a concentration of from about 1 g/L to about 5 g/L; and
recombinant insulin in a concentration of from about 10 mg/L to about 20 mg/L.

4. The method of claim 1, wherein the combined basal medium and supplemental medium include:
antioxidant in a concentration substantially equivalent to a 1× solution of Sigma Cat. No. A-1345,
L-glutamine in a concentration of about 2 mM;
iron in concentration of about 200 mg/L;
ethanolamine in a concentration of about 30 mg/L;
recombinant albumin in a concentration of about 2 g/L; and
recombinant insulin in a concentration of about 15 mg/L.

5. A method of claim 1, wherein the combined basal medium and supplemental medium further includes, in an amount sufficient to support the growth of a single CHO cell, one or more growth factors selected from recombinant epidermal growth factor (EGF) and recombinant insulin like growth factor (IGF).

6. The method of claim 5, wherein the basal medium has substantially the same ingredients as a basal medium selected from the group consisting of VM-Soy and 50/50.

7. The method of claim 5, the combined basal medium and supplemental medium include the following:
an antioxidant in a concentration of from about 0.01 mM to about 10 mM;
L-glutamine in a concentration of from about 2 mM to about 6 mM;
iron in a concentration of from about 10 mg/L to about 400 mg/L;
ethanolamine in a concentration of from about 1 mg/L to about 100 mg/L;
recombinant albumin in a concentration of from about 1 g/L to about 5 g/L;
recombinant insulin in a concentration of from about 10 mg/L to about 20 mg/L;
recombinant EGF, if present, in a concentration of from about 0.01 μg/L to about 100 μg/L; and
recombinant IGF, if present, in a concentration of from about 1 μg/L to about 1500 μg/L.

8. The method of claim 5, wherein the combined basal medium and supplemental medium include:
antioxidant in a concentration substantially equivalent to a 1× solution of Sigma Cat. No. A-1345,
L-glutamine in a concentration of about 2 mM;
iron in concentration of about 200 mg/L;
ethanolamine in a concentration of about 30 mg/L;
recombinant albumin in a concentration of about 2 g/L;
recombinant insulin in a concentration of about 15 mg/L;
recombinant EGF, if present, in a concentration of about 10 μg/L; and
recombinant IGF, if present, in a concentration of about 100 μg/L.

9. A method of culturing a single CHO cell in a serum-free cell culture medium, comprising the steps of:
(a) culturing a population of CHO cells at a cell density greater than about 10⁴ cells/ml in a basal medium sufficient to support the serum-free growth of CHO cells;
(b) isolating a single CHO cell; and
(c) adding to the basal medium a supplemental medium, wherein the combined basal medium and supplemental medium include an antioxidant, L-glutamine, iron, ethanolamine, recombinant albumin, and recombinant insulin, in an amount sufficient to support the growth of a single CHO cell.

10. The method of claim 9, wherein the basal medium has substantially the same ingredients as a basal medium selected from the group consisting of VM-Soy and 50/50.

11. The method of claim 9, wherein the combined basal medium and supplemental medium include:
antioxidant in a concentration of from about 0.01 mM to about 10 mM;
L-glutamine in a concentration of from about 2 mM to about 6 mM;
iron in a concentration of from about 10 mg/L to about 400 mg/L;
ethanolamine in a concentration of from about 1 mg/L to about 100 mg/L;
recombinant albumin in a concentration of from about 1 g/L to about 5 g/L; and
recombinant insulin in a concentration of from about 10 mg/L to about 20 mg/L.

12. The method of claim 9, wherein the combined basal medium and supplemental medium include:
antioxidant in a concentration substantially equivalent to a 1× solution of Sigma Cat. No. A-1345,
L-glutamine in a concentration of about 2 mM;
iron in concentration of about 200 mg/L;
ethanolamine in a concentration of about 30 mg/L;
recombinant albumin in a concentration of about 2 g/L; and
recombinant insulin in a concentration of about 15 mg/L.

13. A method of claim 9, wherein the combined basal medium and supplemental medium further includes, in an amount sufficient to support the growth of a single CHO cell, one or more growth factors selected from recombinant epidermal growth factor (EGF) and recombinant insulin like growth factor (IGF).

14. The method of claim 13, wherein the basal medium has substantially the same ingredients as a basal medium selected from the group consisting of VM-Soy and 50/50.

15. The method of claim 13, the combined basal medium and supplemental medium include, in an amount sufficient to support the growth of a single CHO cell, the following:
an antioxidant in a concentration of from about 0.01 mM to about 10 mM;
L-glutamine in a concentration of from about 2 mM to about 6 mM;
iron in a concentration of from about 10 mg/L to about 400 mg/L;
ethanolamine in a concentration of from about 1 mg/L to about 100 mg/L;
recombinant albumin in a concentration of from about 1 g/L to about 5 g/L;
recombinant insulin in a concentration of from about 10 mg/L to about 20 mg/L;
recombinant EGF, if present, in a concentration of from about 0.01 μg/L to about 100 μg/L; and
recombinant IGF, if present, in a concentration of from about 1 μg/L to about 1500 μg/L.

16. The method of claim 13, wherein the combined basal medium and supplemental medium include:
antioxidant in a concentration substantially equivalent to a 1× solution of Sigma Cat. No. A-1345,
L-glutamine in a concentration of about 2 mM;
iron in concentration of about 200 mg/L;
ethanolamine in a concentration of about 30 mg/L;
recombinant albumin in a concentration of about 2 g/L;
recombinant insulin in a concentration of about 15 mg/L;
recombinant EGF, if present, in a concentration of about 10 μg/L; and
recombinant IGF, if present, in a concentration of about 100 μg/L.

17. A serum-free CHO cell culture medium comprising a basal medium sufficient to support the serum-free growth of CHO cells and a supplemental medium, wherein the combined basal medium and supplemental medium include, in an amount sufficient to support the growth of a single CHO cell, an antioxidant, L-glutamine, iron, ethanolamine, recombinant albumin, and recombinant insulin.

18. The cell culture medium of claim 17, wherein the basal medium has substantially the same ingredients as a basal medium selected from the group consisting of VM-Soy and 50/50.

19. The cell culture medium of claim 17, wherein the combined basal medium and supplemental medium include the following:
antioxidant in a concentration of from about 0.01 mM to about 10 mM;
L-glutamine in a concentration of from about 2 mM to about 6 mM;
iron in a concentration of from about 10 mg/L to about 400 mg/L;
ethanolamine in a concentration of from about 1 mg/L to about 100 mg/L;
recombinant albumin in a concentration of from about 1 g/L to about 5 g/L; and
recombinant insulin in a concentration of from about 10 mg/L to about 20 mg/L.

20. The cell culture medium of claim 17, wherein the combined basal medium and supplemental medium include the following:
antioxidant in a concentration substantially equivalent to a 1× solution of Sigma Cat. No. A-1345,
L-glutamine in a concentration of about 2 mM;
iron in concentration of about 200 mg/L;
ethanolamine in a concentration of about 30 mg/L;
recombinant albumin in a concentration of about 2 g/L; and
recombinant insulin in a concentration of about 15 mg/L.

21. The cell culture medium of claim 17, wherein the combined basal medium and supplemental medium further includes, in an amount sufficient to support the growth of a single CHO cell, one or more growth factors selected from recombinant epidermal growth factor (EGF) and recombinant insulin like growth factor (IGF).

22. The cell culture medium of claim 21, wherein the basal medium has substantially the same ingredients as a basal medium selected from the group consisting of VM-Soy and 50/50.

23. The cell culture medium of claim 21, the combined basal medium and supplemental medium include the following:
antioxidant in a concentration of from about 0.01 mM to about 10 mM;
L-glutamine in a concentration of from about 2 mM to about 6 mM;
iron in a concentration of from about 10 mg/L to about 400 mg/L;
ethanolamine in a concentration of from about 1 mg/L to about 100 mg/L;
recombinant albumin in a concentration of from about 1 g/L to about 5 g/L;
recombinant insulin in a concentration of from about 10 mg/L to about 20 mg/L;
recombinant EGF, if present, in a concentration of from about 0.01 μg/L to about 100 μg/L; and
recombinant IGF, if present, in a concentration of from about 1 μg/L to about 1500 μg/L.

24. The cell culture medium of claim 21, wherein the combined basal medium and supplemental medium include the following:
antioxidant in a concentration substantially equivalent to a 1× solution of Sigma Cat. No. A-1345,
L-glutamine in a concentration of about 2 mM;
iron in concentration of about 200 mg/L;
ethanolamine in a concentration of about 30 mg/L;
recombinant albumin in a concentration of about 2 g/L;
recombinant insulin in a concentration of about 15 mg/L;
recombinant EGF, if present, in a concentration of about 10 μg/L; and
recombinant IGF, if present, in a concentration of about 100 μg/L.

25. A CHO cell in a culture medium according to claim 1.
